(19) United States
(12) Patent Application Publication
(10) Pub. No.: US 2004/0034022 A1
(43) Pub. Date: Feb. 19, 2004

(54) Polymorphic forms/hydrates of n-[4-(3-chloro-4-fluorophenylamino)-7-(3-morpholin-4-ylpropoxy)-quinazolin-6-yl]-acrylamide dihydrochloride

(76) Inventors: Hubert Barth, Emmendingen (DE); Klaus Steiner, Emmendingen (DE); Simon Schneider, Merzhausen (DE); Dietmar Huls, Freiburg (DE); Andreas Muhlenfeld, Freiburg (DE); Manfred Westermayer, Gundelfingen (DE)

Correspondence Address: Rosanne Goodman, Warner Lambert Company, 2800 Plymounth Road, Ann Arbor, MI, 48105, US

(21) Appl. No.: 10/312,173

(22) PCT Filed: Jun. 15, 2001
(86) PCT No.: PCT/EP01/06733

(30) Foreign Application Priority Data
Jun. 30, 2000 (DE) 100 31 971.8

(51) Int. Cl. A61K 31/5377; C07D 413/02

(52) U.S. Cl. 514/234.2; 544/116

(57) ABSTRACT

There are described polymorphic forms/hydrates of N-[4-(3-chloro-4-fluorophenylamino)-7-(3-morpholin-4-yl-propoxy)-quinazolin-6-yl]-acrylamide dihydrochloride, processes for their preparation, as well as the use of the same for the preparation of medicaments with irreversible tyrosine kinase inhibiting action.

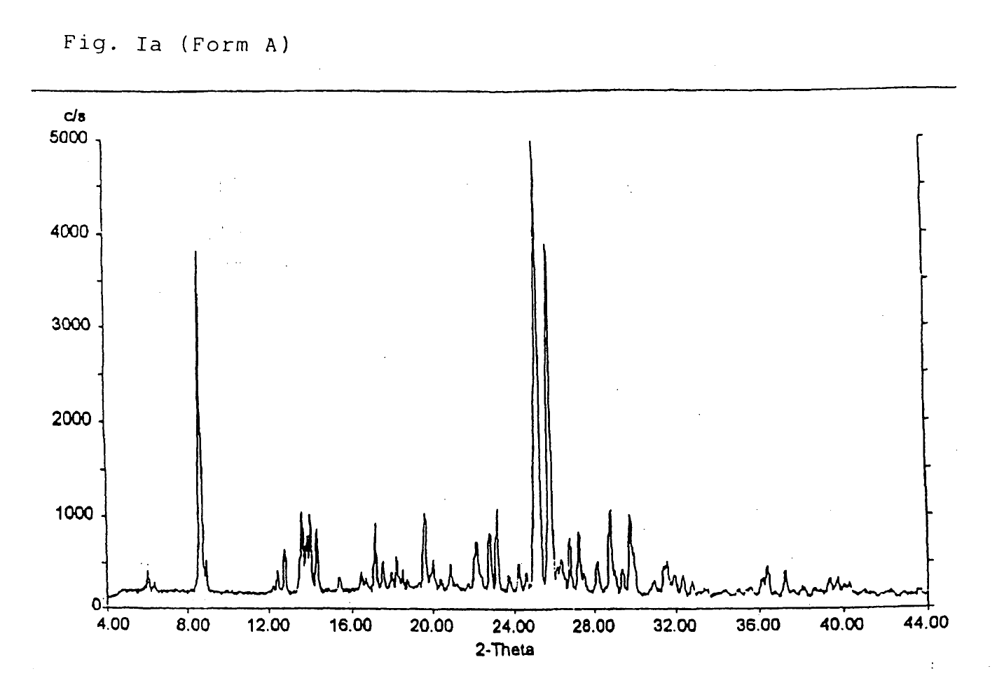

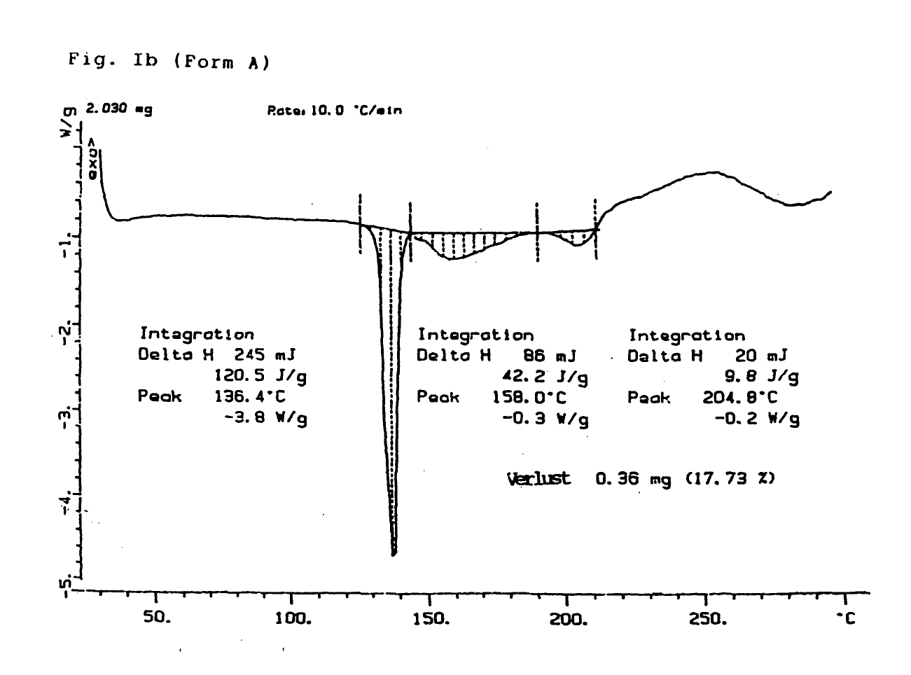

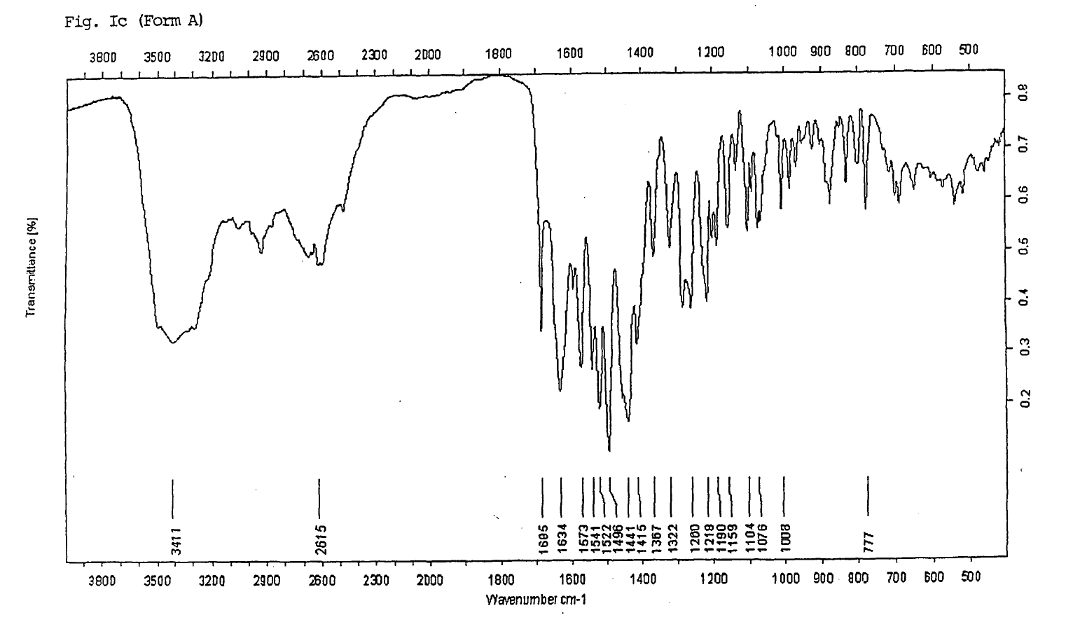

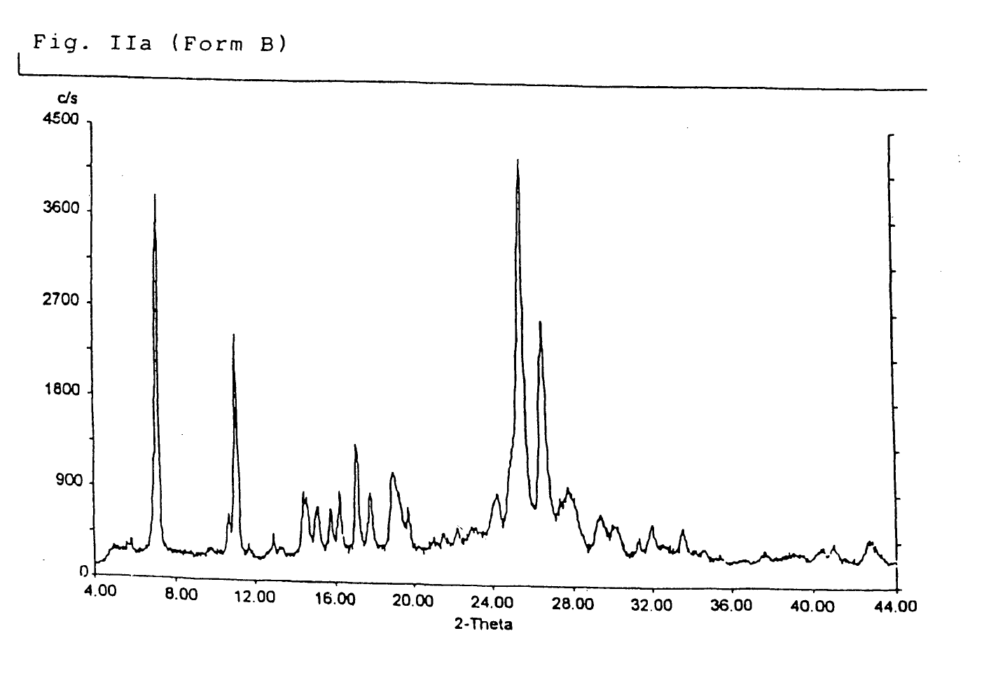

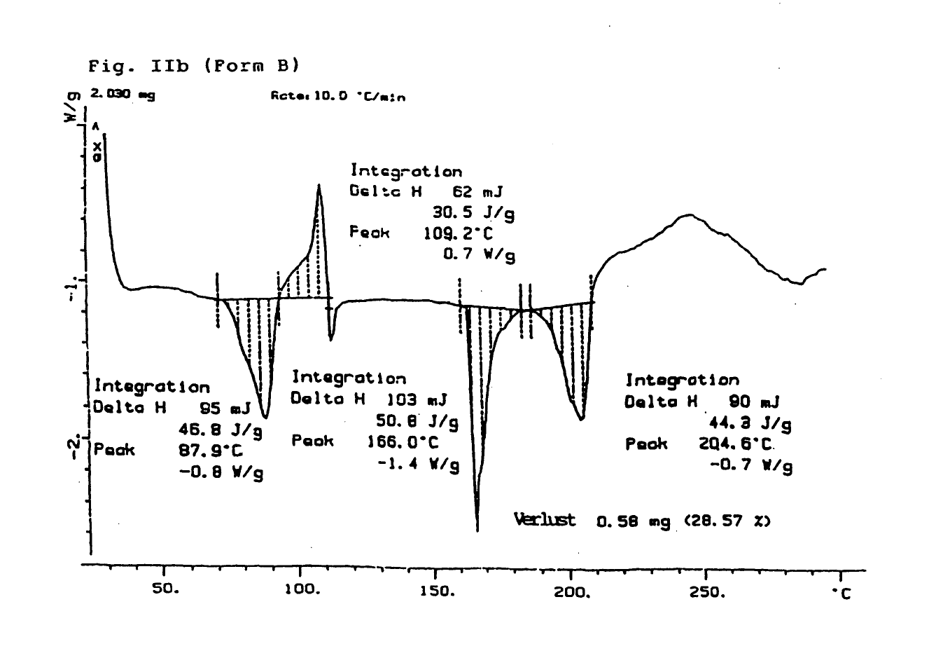

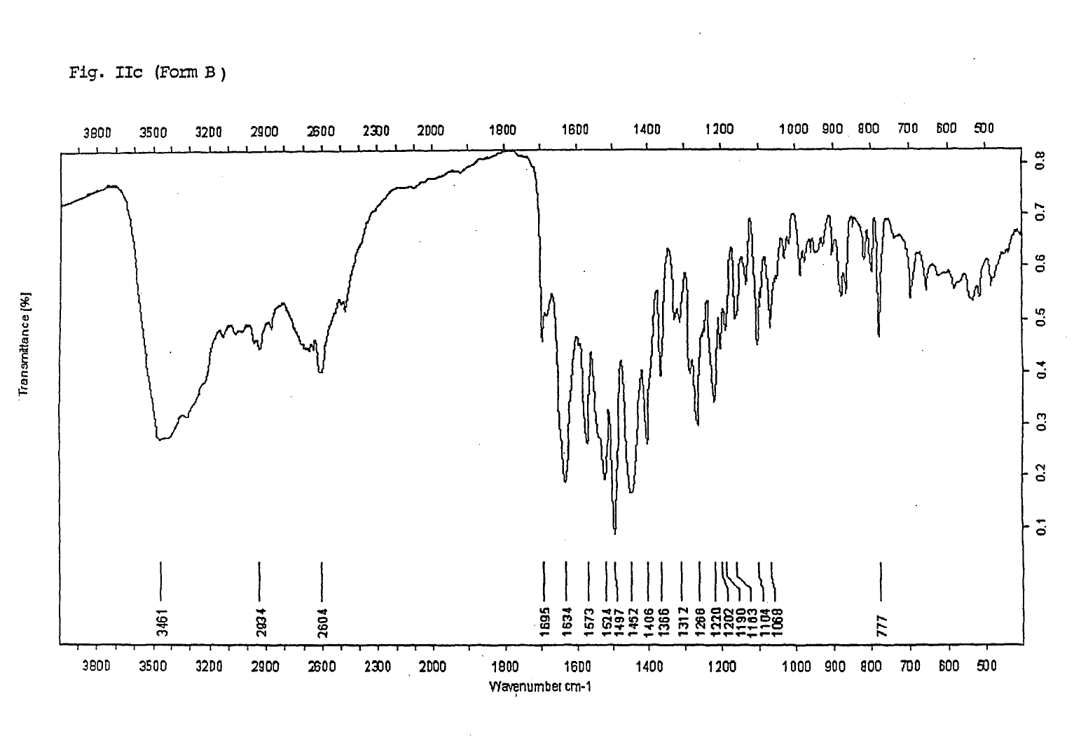

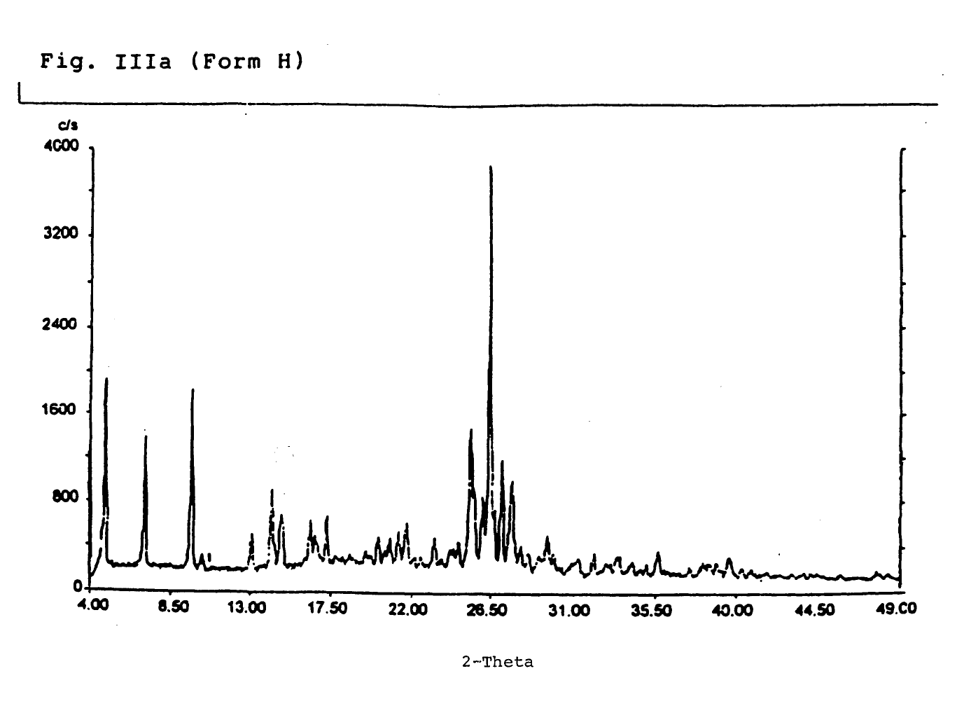

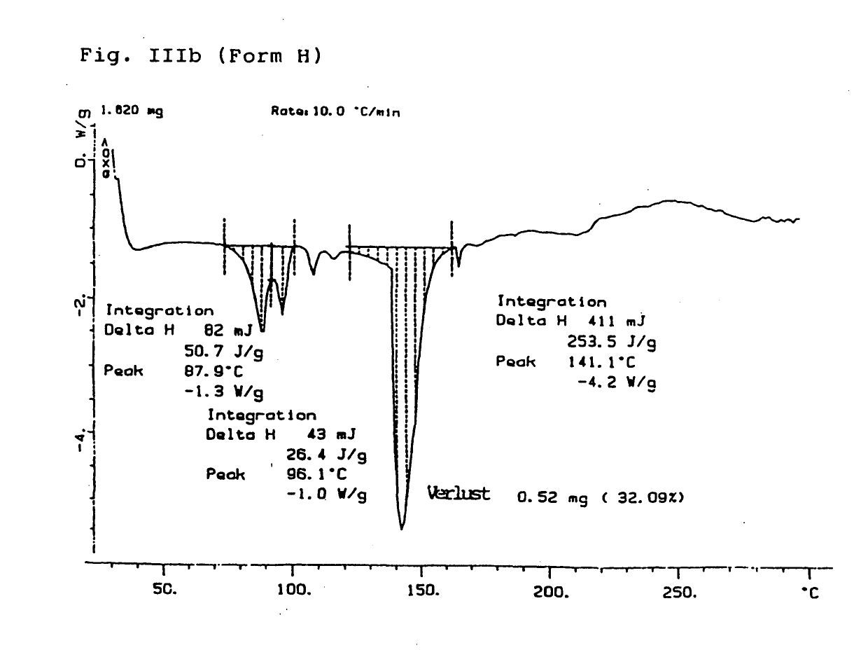

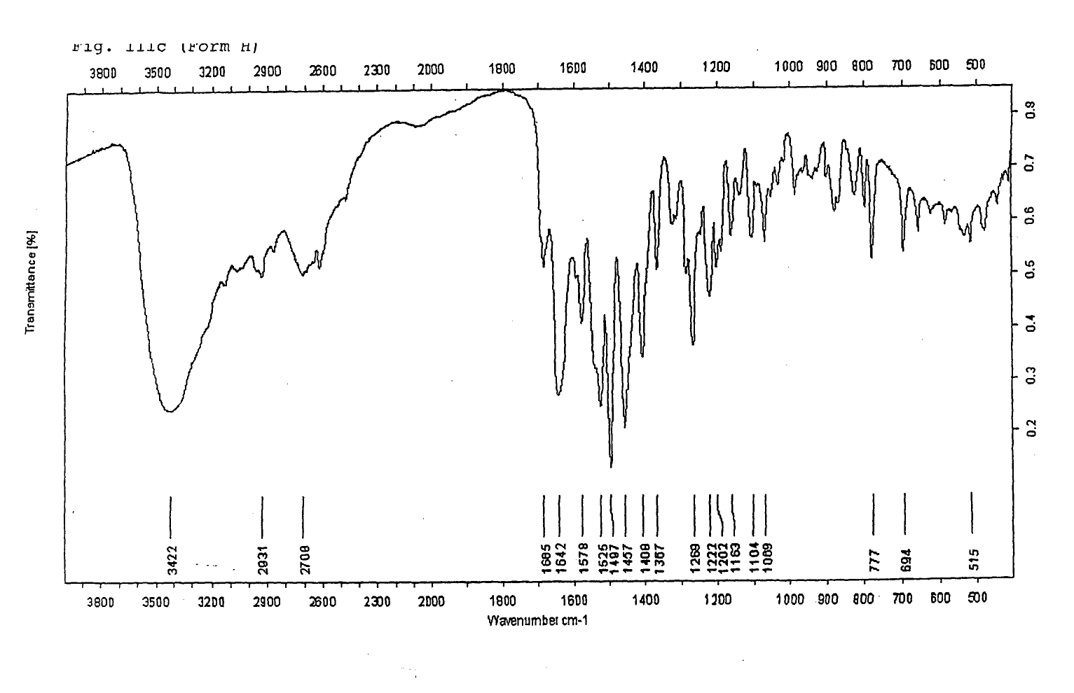

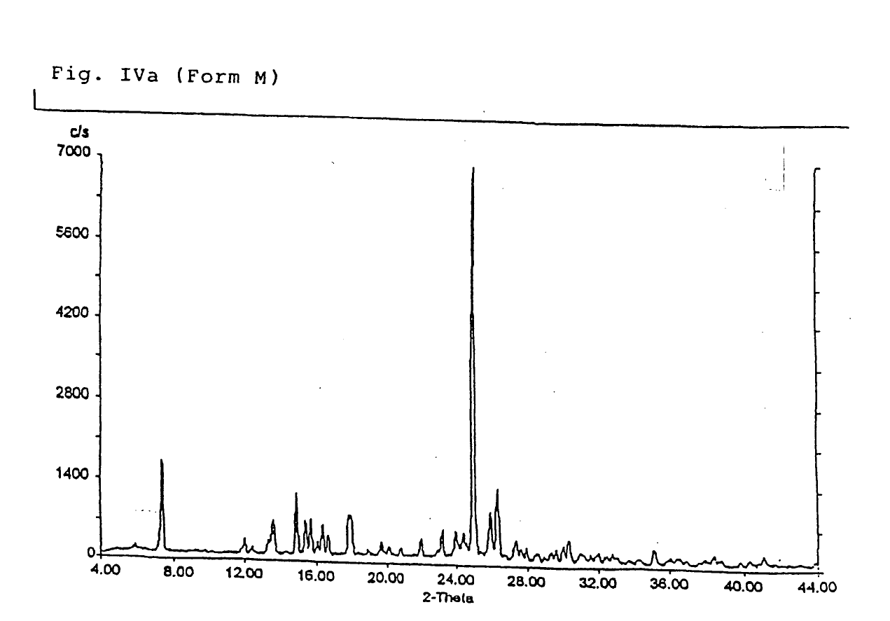

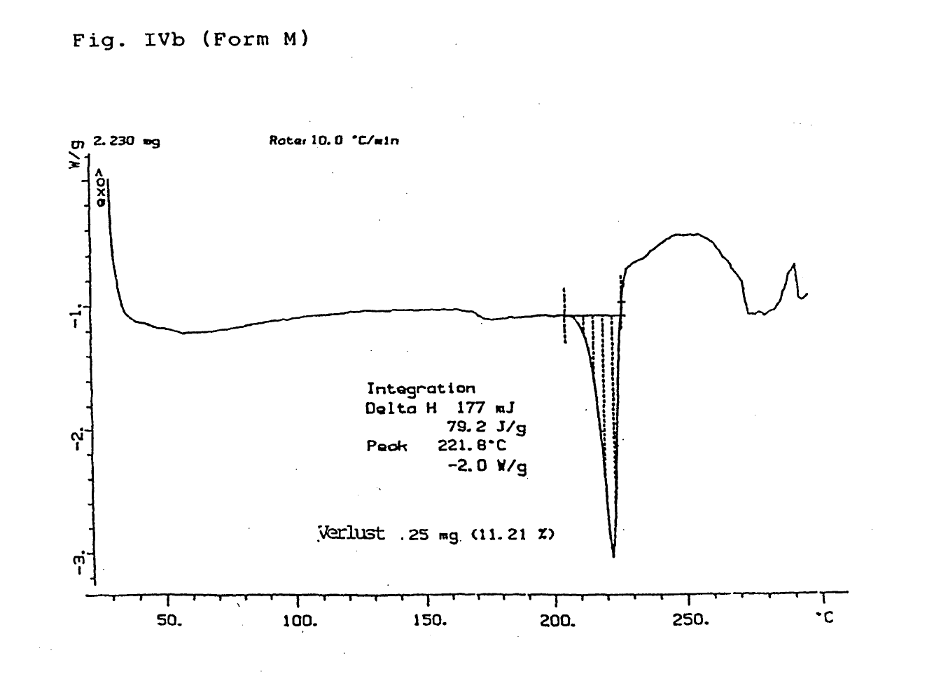

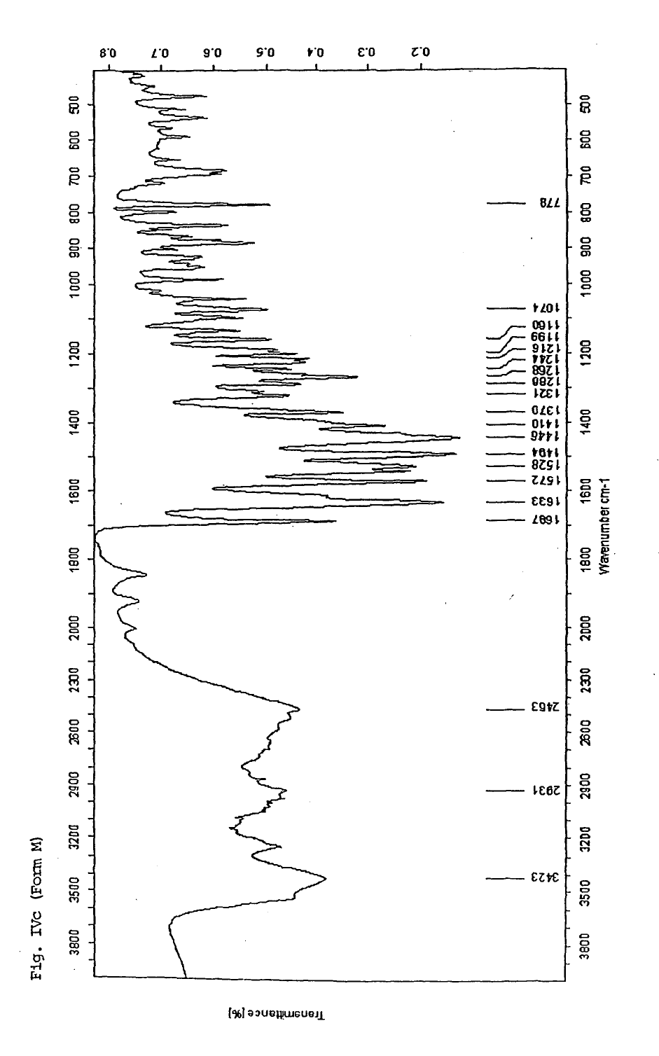

The N-[4-(3-chloro-4-fluorophenylamino)-7-(3-morpholin-4-ylpropoxy)-quinazolin-6-yl]-acrylamide dihydrochloride of the formula:

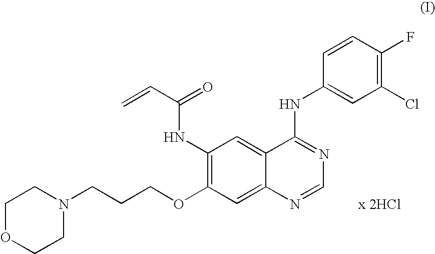

is a representative of a new class of highly effective irreversible tyrosine kinase inhibitors of the EGF receptor which, inter alia, is to be used for the treatment of different tumours (WO-97/38983). The preparation of the corresponding free base is described in the U.S. Patent Application No. 60/109,065 with the application date of Nov. 19, 1998.

It is known that different polymorphic forms/hydrates of an active material can have a strong influence on the stability, the solubility, the formulation properties and the preparation of a medicament.

Furthermore, different polymorphic forms/hydrates of an active material can strongly influence the action itself since various take-up and distribution speeds in the body can have the result of different concentration of the active material at the place of action and thus different biological actions are to be expected.

In the case of the preparation of the compound (I) from the free base, it has, surprisingly, been shown that the compound (I) is able to form different polymorphic forms/hydrates. These differ clearly in their X-ray powder diagrams, differential scanning calorimetry curves and the water values measured according to the Karl Fischer method and—less clearly—by their IR spectra.

In that the different polymorphic forms/hydrates of the compound (I) can be clearly characterised by the mentioned physical determination processes, the above-mentioned fact that the appearance of unknown polymorphic forms/hydrates of an active material exercises a strong influence on the preparation of a medicament, can be taken into account in the case of the formulation of the medicament in question and official conditions (e.g. the conditions of the FDA), according to which no medicaments can be marketed which have been produced with the use of polymorphic forms/hydrates of an active material not clearly characterised by physical or chemical parameters.

In the scope of relevant investigations, four different polymorphic forms/hydrates of the compound (I) have been prepared and characterised, namely, the form A with about=3 mole of water, the form B as polymorphic compound of tine form A also with about 3 mole of water, the form H with about 7 mole of water and the form M with about 1 mole of water.

The characterisation of the different forms A to M of the compound (I) took place from their X-ray powder diagrams, differential scanning calorimetry diagrams and IR spectra, as well as by their water values determined according to the Karl Fischer method and their elementary analysis values. The said diagrams and spectra are illustrated in the drawings.

In detail are shown:

Figures Ia to IVa X-ray powder diagrams of the forms A, B and M of the compound (I);

Figures Ib to IVb differential scanning calorimetry diagrams of the forms A, B, H and M of the compound (I) and

Figures Ic to IVc the IR spectra of the forms A, B, H and M of the compound (I).

In the case of the preparation of the compound (I) from the free base and aqueous hydrochloric acid in a mixture of 20 parts absolute ethanol and 1 part water, the form M of the compound (I) results with about 1 mol water.

If the form M of the compound (I) is crystallised out from a mixture of 10 parts absolute ethanol and 1 Part water, the compound (I) is obtained in the form A with about 3 mol of water. In the case of the crystallising out of the form A of the compound (I) from water and subsequent suitable drying of the crystals obtained, there results a compound polymorphic to form A of the compound (I) which is designated as form B and also contains about 3 mol of water.

The preparation of the compound (I) from the free base and hydrochloric acid in water leads, after suitable drying of the product, to the form B of the compound (I).

If the form B of the compound (I) is dissolved in absolute methanol and the solvent allowed to evaporate at room temperature, the form H of the compound (I) results with about 7 mole of water. The form H can also be obtained by crystallisation of the forms A or B from 1N hydrochloric acid and suitable drying of the crystals obtained.

As already mentioned, the different polymorphic forms/hydrates A, B, H and M of the compound (I) obtained in reproducible ways clearly differ in their X-ray powder diagrams and differential scanning calorimetry diagrams, as well as in the water values according to Karl Fischer, as well as less clearly in their IR spectra. A further difference between the various forms consists in a differing stability towards the heating of the solid substance at 80° C. or 150° C. In comparison to the forms A or B, the form M proves to be the more stable form.

It is to be pointed out that in the preparation of the compound. (I) from the free base and hydrochloric acid, products can also be obtained which, according to X-ray powder diagrams, are mixed forms of A and B and, like the forms A and B themselves, crystallise with a definite water content of 3 mole of water.

The different forms of the compound (I) according to the invention are suitable in the same way as the compound (I) itself for use as irreversible tyrosine kinase inhibitors and thus for the making available of medicaments for the treatment of cancer, arteriosclerosis, restenosis, endometriosis and psoriasis.

The following Examples are to illustrate the invention in more detail but in no way to limit.

### EXAMPLE 1

Preparation of N-[4-(3-chloro-4-fluorophenylamino)-7-(3-morpholin-4-ylpropoxy)-quinazolin-6-yl]-acrylamide Dihydrochloride Form M.

A 6 l three-necked flask equipped wich a mechanical stirrer, a reflux condenser and a dropping funnel is supplied with 300 g N-[4-(3-chloro-4-fluorophenylamino)-7-(3-morpholin-4-ylpropoxy)-quinazolin-6-yl]-acrylamide and 4 l abs. ethanol. With stirring, the suspension is heated to 35° C. A mixture of 100 ml conc. hydrochloric acid and 100 ml water is then added dropwise thereto within 30 s and the reaction mixture further heated to 74° C. At 40° C, a clear solution results, at about 50° C. the solution becomes turbid and the crystallisation commences. With stirring, one allows the reaction mixture to cool slowly to room temperature and then cools further in an icebath for 2 h to 2° C. The precipitated crystals are filtered off with suction and dried in a circulating drying cabinet for 40 h at 60° C. Thereafter, the product is carefully sieved through a 0.5 mm Kressner sieve. One obtains 314.2 g of product.

Water according to Karl Fischer: 2.84%.

Elementary analysis: (C₂₄H₂₅ClFNSO₃×2HCl×H₂O)

| | C | N | N | Cl | F | Cl (ion.) |
|---|---|---|---|---|---|---|
| calc.: | 49.97 | 5.07 | 12.14 | 18.44 | 3.29 | 12.29 |
| found: | 50.08 | 5.18 | 12.08 | 18.38 | 3.15 | 12.20 |

### EXAMPLE 2

Preparation of N-[4-(3-chloro-4-fluorophenylamino)-7-(3-morpholin-4-ylpropoxy)-quinazolin-6-yl]-acrylamide Dihydrochloride Form A

A suspension of 120 g N-[4-(3-chloro-4-fluorophenylamino)-7-(3-morpholin-4-ylpropoxy)-quinazolin-6-yl]-acrylamide dihydrochloride form M and 300 ml of a mixture of 10 parts abs. ethanol and 1 part of water (v/v) is heated, with stirring, to 75° C. The yellowish solution is filtered through a folded filter and the filtrate slowly cooled with stirring. One further stirs for 3 h at room temperature and then for 2 h in an ice-bath. The precipitated product is filtered off with suction and dried for 3 h at 40° C. and 36 h at 60° C. in a circulating drying cabinet. One obtains 10.7 g of product.

Water according to Karl Fischer: 8.82%.

Elementary analysis: (C₂₄H₂₅ClFN₅O₃×2HCl×3H₂O)

| | C | H | N | Cl | F | Cl (ion.) |
|---|---|---|---|---|---|---|
| calc.: | 47.03 | 5.43 | 11.43 | 17.35 | 3.10 | 11.57 |
| found: | 47.05 | 5.30 | 11.47 | 17.50 | 2.98 | 11.53 |

### EXAMPLE 3

Preparation of N-[4-(3-chloro-4-fluorophenylamino)-7-(3-morpholin-4-ylpropoxy)-quinazolin-6-yl]-acrylamide Dihydrochloride Form B

A Suspension of 250 g N-[4-(3-chloro-4-fluorophenylamino)-7-(3-morpholin-4-ylpropoxy)-quinazlon-6-yl]-acrylamide dihydrochloride form A in 2.6 l water is heated, with stirring, to 50° C. The slightly turbid solution is sucked through a Buchner funnel (porosity a) and the filtrate cooled to room temperature without stirring. After standing overnight in a refrigerator at 4° C., the precipitated product is filtered off with suction, washed out with 100 ml water and dried in a vacuum desiccator over calcium chloride at 20 mbar for 3 days. The product obtained is sieved over a 1 mm Kresner sieve. One obtains 212.2 g of product.

Water according to Karl Fischer: 8.6%

Elementary analysis: (C₂₄H₂ClFN₅O₃×2HCl×3H₂O)

| | C | H | N | Cl | F | Cl (ion.) |
|---|---|---|---|---|---|---|
| calc.: | 47.03 | 5.43 | 11.43 | 17.35 | 3.10 | 11.57 |
| found: | 47.28 | 5.35 | 11.50 | 17.47 | 2.94 | 11.32 |

### EXAMPLE 4

Preparation of N-[4-(3-chloro-4-fluorophenylamino)-7-(3-morpholin-4-ylpropoxy)-quinazolin-6-yl]-acrylamide Dihydrochloride Form H

2 g N-[4-(3-Chloro-4-fluorophenylamino)-7-(3-morpholin-4-ylpropoxy)-quinazolin-6-yl]-acrylamide dihydrochloride form B are dissolved in 80 ml abs. methanol at room temperature. The solution is filtered into a crystallisation dish and kept open under an extractor up to the complete evaporation of the solvent (7 days).

Water according to Karl Fischer: 19.95%

Elementary analysis: (C₂₄H₂ClFNFO₃×2HCl×7H₂O)

| | C | H | N | Cl | F | Cl (ion.) |
|---|---|---|---|---|---|---|
| calc.: | 42.08 | 6.03 | 10.22 | 15.53 | 2.77 | 10.35 |
| found: | 42.16 | 6.20 | 10.24 | 15.76 | 2.68 | 10.11 |

### EXAMPLE 5

N-[4-(3-chloro-4-fluorophenylamino)-7-(3-morpholin-4-ylpropoxy)-quinazolin-6-yl]-acrylamide Dihydrochloride Form H

Form H can also be obtained as follows by crystallisation of form B from 1N hydrochloric acid:

A suspension of 1 g N-[4-(3-chloro-4-fluorophenyl-amino)-7-(3-morpholin-4-ylpropoxy)-quinazolin-6-yl]-acrylamide dihydrochloride form B and 20 ml 1N hydrochloric acid is heated with stirring to 60° C. One allows the filtered solution to cool to room temperature with stirring and then keeps it overnight in a refrigerator at 4° C. The precipitated product is filtered off with suction, washed out with a little water and, after comminution and transferal into a crystallisation dish, dried for 2 days at room temperature in the open dish in the air.

### EXAMPLE 6

Temperature Stress Experiments

For the investigation of the thermal stability, the solid forms A, B and M are heated in open test tubes (l: 110 mm, d: 5 mm) or in test tubes closed by means of a glass stamp in an oil bath at temperatures and for a period of time as given in the Table. Subsequently, the purity of the remaining products is investigated by means of HPLC methods (column: LunaRP18 (25×0.46 cm); mobile phase: acetonitrile:methanol: 0.02M aq. ammonium acetate:triethylamine (55:5:40:0.05).

Temperature Stress

| | HPLC Purity | Stress | HPLC Purity |
|---|---|---|---|
| Form | Start [rel %] | Conditions | [rel %] |
| A | 99.28 | 7 d, 80° C. | 97.51 |
| | | | 99.02 (s) |
| | | 16 hr, 100° C., | 25.28 |
| | | then 8 hr, | 23.45 (s) |
| | | 150° C. | |
| B | 99.77 | 7 d, 80° C. | 89.39 |
| | | | 84.40 (s) |
| | | 16 hr, 100° C., | 75.32 |
| | | then | 75.28 (s) |
| | | 8 hr, 150° C. | |
| M | 99.49 | 8 d, 80° C. | 99.62 |
| | | | 99.61 (s) |
| | | 16 hr, 100° C., | 99.43 |
| | | then | 99.43 (s) |
| | | 8 hr, 150° C. | |

(s): test tube with glass stamp

1. Polymorphic forms/hydrates of N-[4-(3-chloro-4-fluorophenylamino)-7-(3-morpholin-4-ylpropoxy)-quinazolin-6-yl]-acrylamide dihydrochloride corresponding to the following formula

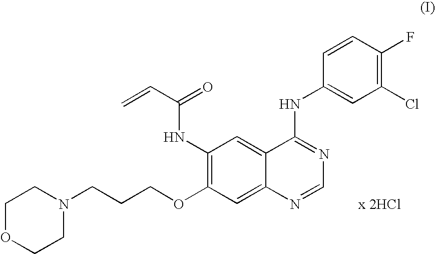

2. Form A of the dihydrochloride according to claim 1 containing about 3 mol of water.

3. Form B of the dihydrochloride according to claim 1 as polymorphic compound to form A according to claim 2 also with about 3 mole of water.

4. Form H of the dihydrochloride according to claim 1 containing about 7 mole of water.

5. Form M of the dihydrochloride according to claim 1 containing about 1 mole of water.

6. Form A of the dihydrochloride according to claim 1 and 2, characterised by diffraction peaks 2 Θ in the X-ray powder diagram at 8.7760°, 23.2083°, 28.8604°, 37.2905°.

7. Form A of the dihydrochloride according to claim 6 additionally characterised by a differential scanning calorimetry diagram according to Fig. Ib.

8. Form B of the dihydrochloride according to claim 1 and 3 as polymorphic compound of form A according to claim 2, characterised by diffraction peaks 2 Θ in the X-ray powder diagram at 11.0986°, 19.0075°, 25.5280°.

9. Form B of the dihydrochloride according to claim 8 additionally characterised by a differential scanning calorimetry diagram according to Fig. IIb.

10. Form M of the dihydrochloride according to claim 1 and 4, characterised by diffraction peaks 2 Θ in the X-ray powder diagram at 7.4267°, 12.0027°, 24.9997°, 35.1642°.

11. Form H of the dihydrochloride according to claim 10, additionally characterised by a differential scanning calorimetry diagram according to Fig. IIIb.

12. Form M of the dihydrochloride according to claim 1 and 5, characterised by diffraction peaks 2 Θ in the X-ray powder diagram at 4.8985°, 9.7296°, 27.1578°, 35.7101°.

13. Form M of the dihydrochloride according to claim 12, additionally characterised by a differential scanning calorimetry diagram according to Fig. IVb.

14. Process for the preparation of the form A of the dihydrochloride according to claim 2 from the free base N-[4-(3-chloro-4-fluorophenylamino)-7-(3-morpholin-4-yl-propoxy)-quinazolin-6-yl]-acrylamide made available in the usual way and aqueous hydrochloric acid, characterised in that the reaction is carried out in a mixture of 20 parts absolute ethanol and 1 part of water for the formation of the form M and the formed form M is crystallised out from a mixture of 10 parts absolute ethanol and 1 part of water.

15. Process for the preparation of the form B of the dihydrochloride according to claim 3 as polymorphous compound to form A according to claim 2, characterised in that the form A obtained according to claim 14 is crystallised from water and the crystals obtained are dried in suitable way.

16. Process for the preparation of the form B of the dihydrochloride according to claim 3 as polymorphic compound to form A according to claim 2 by preparation of the dihydrochloride from the free base N-[4-(3-chloro-4-fluorophenylamino)-7-(3-morpholin-4-ylpropoxy)-quinazolin-6-yl]-acrylamide made available in usual known way and hydrochloric acid in water as well as suitable drying of the dihydrochloride obtained.

17. Process for the preparation of the form H of the dihydrochloride according to claim 4 by a) dissolving in absolute ethanol of the form B obtained according to the process according to claim 15 or 16 and leaving the ethanol to evaporate at room temperature or b) dissolving and crystallising of the form A obtained according to claim 14 or of the form B obtained according to claim 15 or 16 in or from 1N hydrochloric acid and suitable drying of the crystals obtained.

18. Process for the preparation of the form M of the dihydrochloride according to claim 5 from the free base N-[4-(3-chloro-4-fluorophenylamino)-7-(3-morpholin-4-yl-propoxy)-quinazolin-6-yl]-acrylamide made available in usual known manner and aqueous hydrochloric acid, characterised in that the reaction is carried out in a mixture of 20 parts absolute ethanol and 1 part of water.

19. Form A of the dihydrochloride according to laim 2 obtainable according to the process according to claim 14.

20. Form B of the dihydrochloride according to claim 3 as polymorphic compound to form A according to claim 2 obtainable according to the process according to claim 15 or 16.

21. Form H of the dihydrochloride according to claim 4 obtainable according to the process according to claim 17

22. Form M of the dihydrochloride according to claim 5 obtainable according to the process according to claim 18.

23. Use of one of the dihydrochloride forms A, B, H and/or M according to one of claims 1 to 13 or 19 to 22, possibly together with usual carriers or adjuvants, for the preparation of a medicament with irreversible tyrosine kinase inhibition action.
